(19) United States
(12) Patent Application Publication
(10) Pub. No.: US 2026/0345673 A1
(43) Pub. Date: Nov. 5, 2026

(54) Apparatus and Method for Contact Free Delivery and Administration of Magnesium as a Drug

(71) Applicant: Yon Doo Ough, Seal Beach, CA (US)

(72) Inventor: Yon Doo Ough, Seal Beach, CA (US)

(21) Appl. No.: 19/483,567

(22) PCT Filed: Jun. 14, 2024
(86) PCT No.: PCT/US2024/034100

§ 371 Date: Nov. 11, 2025

Related U.S. Application Data
(63) Continuation-in-part of application No. 18/486,768, filed on Oct. 13, 2023.
Application No. 18/486,768 is a continuation of application No. PCT/US2023/076887, filed on Oct. 13, 2023.
(60) Provisional application No. 63/501,592, filed on May 11, 2023.

(51) Int. Cl. A61K 33/06; A61K 9/00; A61K 33/00; A61K 47/38; A61K 47/44

(52) U.S. Cl. CPC A61K 33/06; A61K 9/0087; A61K 33/00; A61K 47/38; A61K 47/44

(57) ABSTRACT

Muscle, tendon, and vessel spasm, cramping, inflammation and related symptoms are treated with high doses of magnesium without ingestions, injection, or topical application. A therapeutic patty is formed with a desired load of magnesium (for example magnesium powder) combined with an emulsifier, such as microcrystalline cellulose, and plant oil. The combination is mechanically stirred to form a firm patty and then shaped into a desired form. The patty may be wrapped in various fabric. In use, the patty is placed near a patient's skin, though not necessarily in physical contact, to provide the therapeutic effect.

### TECHNICAL FIELD OF THE INVENTION

The present invention relates to medical treatment in the field of muscle, tendon, and vessel spasm, cramping, inflammation and related symptoms. In various embodiments, the present invention relates more particularly to a contact free method of treating muscle pain, spasms, and cramping with large doses of magnesium placed near a patient's body. The present invention also relates to a sleep aid for promoting sound, deep, and natural sleep. In general, the present invention discloses the use of a therapeutic compound, such as magnesium, that is delivered to a patient in a desirable dose but without any physical contact or actual consumption of the therapeutic compound by the patient (e.g., without ingestion, injection, or topical application). Consequently, the risk of side effects or adverse reactions to the therapeutic compound are minimized or eliminated and the therapeutic compound can be used and reused over an extended period of time without loss of potency.

### BACKGROUND

Magnesium has been known to be a safe treatment for various muscle related maladies. For example, magnesium hydroxide is prescribed for treating constipation. Additionally, magnesium sulfate is prescribed for pre-eclamptic patients to prevent development of seizures. For treatment of preeclampsia and eclampsia, a patient may be given an initial dose of 4-6 grams of magnesium sulfate intravenously over a period of 20 minutes followed by a maintenance dose of 1-3 grams/hour intravenously. The patient may also be given intramuscular injections of magnesium sulfate. The maximum safe daily dose of magnesium sulfate delivered intravenously is 60-70 grams/day.

Magnesium sulfate has also been used to treat and prevent premature labor contractions, thereby preventing premature labor by relaxing contraction (e.g., spasms) of uterine smooth muscles. A patient experiencing premature labor contractions may be given large doses of magnesium sulfate for a period of 5-7 days or until satisfactory results are achieved. In advanced cardiac life support, magnesium sulfate may be given intravenously at a dosage of 1 gram in a diluted solution over 5-20 minutes.

While magnesium may be contraindicated in various conditions or patients, such as in the presence of hypersensitivity, diabetic coma, heart block, hyper-magnesia, or hyper-calcemia, the uses described above demonstrate that magnesium is generally safe, even in high-risk patient's such as late term pregnant patients. Thus, in the absence of a contraindicator, magnesium is generally considered safe. The only specific form of magnesium that is currently governed by and approved by the U.S. Food and Drug Administration (FDA) is the use of Milk of Magnesia as a laxative to treat constipation. Milk of Magnesia is classified by the FDA as an over-the-counter drug for treatment of constipation. Accordingly, except for obstetric care, magnesium can be safely used by the general public without a physician's prescription. In particular, magnesium hydroxide is classified by the FDA as GRAS (generally recognized as safe).

### SUMMARY OF THE INVENTION

It has been discovered that magnesium has other benefits not previously known in the medical or related fields when provided in extremely large volumes in the manner described herein. While traditional applications of magnesium have used relatively low doses, the current invention uses doses that are as much as 10-600 times larger, and more typically in a range of 100-1,000 grams.

For example, the largest individual doses of magnesium traditionally administered are less than 10 grams of magnesium sulfate, such as the 4-6 grams of magnesium sulfate administered to preeclamptic or eclamptic patients in each hourly treatment. Because the magnesium is administered in the form of a salt (elemental magnesium does not form naturally and thus magnesium always presents in a salt) the actual volume of pure elemental magnesium being administered is less than the dosage of the drug being administered. For example, in the instance of magnesium sulfate, the equivalent volume of pure elemental magnesium being administered is approximately 20% of the total molecular weight. Thus, a dosage of 4-6 grams of magnesium sulfate will deliver only approximately 0.8-1.2 grams of pure magnesium. The maximum safe dose of magnesium sulfate delivered through intravenous injections is 60-70 grams in a 24 hour period, which translates to approximately 12-14 grams of elemental magnesium.

The doses described above are significantly large in comparison to other traditional uses and notably would be extremely large for an oral administration. For example, milk of magnesia is an over-the-counter laxative often used to treat constipation. A standard 30 ml dose of milk of magnesia may only contain 1.2 grams of magnesium hydroxide, which would contain only 0.48 grams of pure elemental magnesium.

Magnesium is also a dietary supplement. Magnesium is an essential mineral and is a cofactor in more than 300 enzyme systems of the human body. Magnesium ions are also required in various catalytic actions within the body including the biological activation of ATP (i.e., adenosine triphosphate) and DNA and RNA synthetization. While most dietary magnesium is obtained through food, it can be supplemented with over-the-counter dietary supplements. Most dietary supplements contain between 0.2 and 0.6 grams of magnesium salts (such as magnesium chloride). The recommended daily allowance of dietary magnesium is between 0.3 and 0.4 grams.

A common characteristic of the above-described delivery methods is the relatively low volumes of magnesium delivered and the limitations of administering higher doses. For example, it would be difficult and dangerous to consume 10's of grams of magnesium orally due to the sheer volume of magnesium that would need to be consumed (either as a powder of combined with other ingredients, which would further increase the volume), and the health risks of administering magnesium through that method. Likewise, taking such large doses of magnesium orally would have a resulting laxative effect.

There would also be a high risk of death for this delivery method and, accordingly, there are no magnesium drugs listed in the FDA monograph and Hospital Drug Formulary. Likewise, it would take a long time to inject 10's of grams of magnesium into a patient in a safe manner and even then the patient would need to be in a hospital setting, in particular in an intensive care unit, where they could be closely monitored for potential complications.

Even with the known localized delivery mechanisms, known methods are not practical for delivering high doses of magnesium. According to the National Institutes of Health, the maximum volume generally acceptable for a subcutaneous injection is around 1.5 ml, which is roughly 60 mg of magnesium sulfate in available injection formulas. Thus, it is not practical or safe to consider delivering high doses of magnesium, for example 40 grams, via a subcutaneous injection. For similar reasons, it would not be practical to deliver high doses of magnesium via topical spray.

The present invention describes and claims a new and safe delivery method for large doses of magnesium that can be delivered without skin contact or oral consumption by the patient. In comparison to a high dose delivered intravenously, which can only be administered in an intensive care unit of a hospital under close observation, the present delivery mechanism delivers a larger measure of magnesium to a desired treatment area and does so in a safe manner. In various exemplary embodiments, a pad delivery apparatus permits the application of very high concentrations and large doses of magnesium without irritation or negative side effect because no magnesium is consumed by the patient and there is no direct contact between the magnesium and the patient's skin.

In various exemplary embodiments, a pad delivery apparatus is comprised of a soft, semi-permeable and breathable fabric that is non-irritating if it comes into contact with a patient's skin. Though in various exemplary embodiments, the pad would not be intended to make contact with the patient's skin, incidental contact may nevertheless occur. A desired load of magnesium is prepared in a pad form by combining magnesium powder, microcrystalline cellulose, and plant oil and mechanically stirring the mixture to form a firm patty. The patty is then wrapped in the soft, semi-permeable and breathable fabric. It should be appreciated that in addition to or in place of microcrystalline cellulose (e.g., refined wood pulp), other appropriate emulsifiers may be used. As used herein, microcrystalline cellulose should be understood to include such other emulsifiers used in addition to or in place of the microcrystalline cellulose.

In various other exemplary embodiments, plant oil is added to the mixture before stirring. In various exemplary embodiments, the volume or weight of magnesium powder used dictates the volume of the other ingredients. For example, microcrystalline cellulose may be provided at 1/20th the volume of magnesium being used and plant oil may be provided at ⅕th the volume of magnesium being used. Comparable measurements and ratios may be provided based on weight of desired magnesium powder.

In each of the various exemplary embodiments described above, once the patty is wrapped in the soft, semi-permeable and breathable fabric, the pad delivery apparatus is placed near the patient's skin but not necessarily in direct physical contact with the patient. Therapeutic effects are typically felt by the patient within a few minutes. It should be appreciated that, while direct contact with the patient's skin is not intended and may not be desired, incidental contact may occur as the patient and/or the therapeutic pad are moved around.

It should be appreciated that other metallic compounds may be used with a pad delivery apparatus as described. For example, lithium, gold, iron, silver, copper, or other elements may be used in place of the magnesium powder.

### BRIEF DESCRIPTION OF THE FIGURES

FIG. 1 is a flow chart showing the method of preparing a therapeutic patty according to the present invention.

### DETAILED DESCRIPTION OF EXEMPLARY EMBODIMENTS

Applying large volumes of magnesium next to a patient's skin (though not necessarily in direct physical contact) is a new and safe way to administer magnesium and it makes it possible to realize full therapeutic effects of magnesium without complications.

It is theorized that vaporized particles of magnesium element are present because of the method of preparing a pad delivery apparatus as described herein and that the vaporized magnesium readily penetrates the patient's body and exerts its therapeutic benefits. For maximum effectiveness, large volumes of magnesium are used, e.g., as much as 600 times the dose of magnesium orally administered when a patient takes milk of magnesia. In such embodiments, any presence of vaporized magnesium, while small in comparison to the overall size of the delivery pad mechanism, may constitute a large dosage of magnesium.

In various exemplary embodiments, a pad delivery apparatus is usable as a sleep aid. In various ones of these exemplary embodiments, a pad delivery apparatus is prepared as described herein and placed in the patient's bed before going to sleep. By simply sleeping in close proximity to the pad delivery apparatus, the patient will experience a sound, deep, and natural sleep. It should be appreciated that while direct contact with the pad may not be intended, incidental contact may occur as the patient moves while sleeping.

In various exemplary embodiments, a pad delivery apparatus is usable to treat nocturnal leg cramps. In various ones of these exemplary embodiments, a pad delivery apparatus is prepared as described herein and placed on the patient's calf muscle or a desirable portion of the patient's leg before sleep. In various exemplary embodiments, the pad delivery apparatus may make at least intermittent physical contact with the patient's leg. In various exemplary embodiments, the pad delivery apparatus may be placed in direct physical contact with the patient's leg, though the patty contained in the pad delivery apparatus need not necessarily make physical contact with the patient. In various exemplary embodiments, the pad delivery apparatus may be physically separated from the patient's skin.

In various exemplary embodiments, a pad delivery apparatus is usable to treat menstrual cramps. In various ones of these exemplary embodiments, a pad delivery apparatus is prepared as described herein and placed on or near the patient's lower abdomen, just below the umbilicus or lower back. Patient's will typically experience a cessation of cramps within 5 minutes.

It should be appreciated that the dose of magnesium used in a pad delivery apparatus may vary depending on the malady or symptom being treated or the desired therapeutic effect. In an exemplary embodiment usable for treatment of muscle cramps and spasms, a pad delivery apparatus is prepared with approximately 50 grams of pure elemental magnesium. In an exemplary embodiment usable as a sleep aide, a pad delivery apparatus is prepared with the equivalent of approximately 250 grams of pure elemental magnesium.

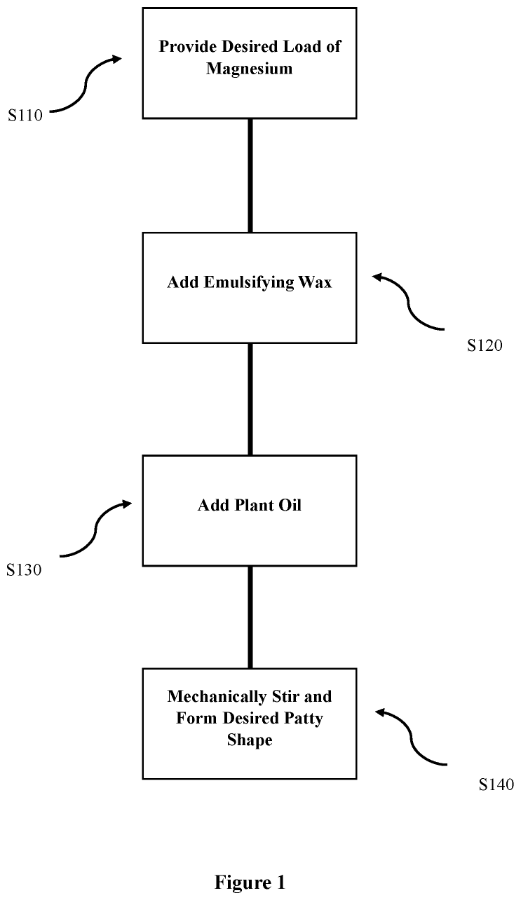

FIG. 1 shows an exemplary method of making a pad delivery apparatus. In step S110, a desired therapeutic dose of magnesium (typically in the form of magnesium powder) is provided. In step S120, a measure of microcrystalline cellulose is added. In various exemplary embodiments, the measure of microcrystalline cellulose is equal to approximately 1/20th the volume of the therapeutic dose of magnesium powder used in step S110. In step S130, a measure of plant oil is added. In various exemplary embodiments, the measure of plant oil is equal to approximately ⅕th the volume the therapeutic dose of magnesium powder used in step S110. At step S140, the ingredients are mechanically stirred until a firm patty forms. The firm patty is formed into a desired shape and wrapped in a soft, semi-permeable and breathable fabric to form the pad delivery apparatus. It should be appreciated that various plant oils may be used. In some embodiments, the plant oil may be a combination of multiple plant oils. Such plant oils may include, for example only, olive oil, coconut oil, palm oil, or any suitable replacements.

1. A method of preparing a pad delivery apparatus comprising:
providing a desired dose of a therapeutic element;
combining the desired dose of the therapeutic element with an emulsifier, and plant oil to form a mixture;
mechanically stirring the mixture until a firm patty forms; and
wrapping the firm patty with a fabric layer.

2. The method of claim 1, wherein the therapeutic element is magnesium.

3. The method of claim 1, wherein the therapeutic element is lithium.

4. The method of claim 1, wherein the emulsifier is microcrystalline cellulose.

5. The method of claim 4, wherein the microcrystalline cellulose used in the combining step is provided in a measure that is approximately 1/20th the volume of the desired dose of the therapeutic element.

6. The method of claim 1, wherein the plant oil used in the combining step is provided in a measure that is approximately ⅕th the volume of the desired dose of the therapeutic element.

7. The method of claim 1, wherein the desired dose of therapeutic compound is determined based on a physical malady to be treated and the patty is placed near a patient's skin, but without direct contact with the patient's skin, for treatment of the physical malady.

8. The method of claim 7, wherein the physical malady is selected from the group consisting of muscle cramps, spasms, tightening, bands, and knots; myofascial pain syndrome; fibromyalgia; constipation; tooth ache; and ankle sprains.

9. The method of claim 7, wherein the physical malady is a sleeping disorder and the patty is usable as a sleeping aid.

10. The method of claim 7, wherein the physical malady is menstrual cramps.

11. The method of claim 7, wherein the physical malady is restless leg syndrome.

12. The method of claim 7, wherein the physical malady is back pain associated with spinal stenosis or a herniated disc.

13. The method of claim 7, wherein no discernable volume of the therapeutic compound is consumed by the patient during treatment.

14. A method of preparing a pad delivery apparatus comprising:
providing a magnesium salt containing approximately 50 grams of pure elemental magnesium;
combining the magnesium salt with an emulsifier and plant oil to form a mixture;
mechanically stirring the mixture until a firm patty forms; and
wrapping the firm patty with a fabric layer.

15. The method of claim 14, wherein the emulsifier is microcrystalline cellulose.

16. The method of claim 14, wherein the combining step further comprises providing the microcrystalline cellulose in a measure that is approximately 1/20th the volume of the magnesium salt and providing the plant oil in a measure that is approximately ⅕th the volume of the magnesium salt.

17. The method of claim 14, wherein the pad delivery apparatus is usable for the treatment of muscle spasms.

18. A method of preparing a pad delivery apparatus comprising:
providing approximately 500 grams of magnesium salt;
combining the magnesium salt with an microcrystalline cellulose and plant oil to form a mixture;
mechanically stirring the mixture until a firm patty forms; and
wrapping the firm patty with a fabric layer.

19. The method of claim 18, wherein the combining step further comprises providing the microcrystalline cellulose in a measure that is approximately 1/20th the volume of the magnesium salt and providing the plant oil in a measure that is approximately ⅕th the volume of the magnesium salt.

20. The method of claim 19, wherein the pad delivery apparatus is usable as a sleeping aid.
